(19) United States
(12) Patent Application Publication
(10) Pub. No.: US 2008/0305191 A1
(43) Pub. Date: Dec. 11, 2008

(54) Drinking Water For a Diabetic and Manufacturing Process Thereof

(76) Inventors: Heon Sang Jeong, Gaesin-dong (KR); Gil Nam Park, Chupungnyeong-myeon (KR)

Correspondence Address: GREER, BURNS & CRAIN, 300 S WACKER DR, 25TH FLOOR, CHICAGO, IL, 60606, US

(21) Appl. No.: 11/883,085

(22) PCT Filed: Feb. 3, 2006
(86) PCT No.: PCT/KR2006/000409

§ 371 Date: Apr. 17, 2008

(30) Foreign Application Priority Data
Feb. 4, 2005 (KR) 10-2005-0010835
Feb. 1, 2006 (KR) 10-2006-0009650

(51) Int. Cl. A61K 36/899; A61K 36/9068; A61K 36/484; A61P 3/10

(52) U.S. Cl. 424/750; 424/756; 424/757

(57) ABSTRACT

A drinking water for a diabetic including water of 93˜97 wt %; budded Oryza sativa of 2.5˜5 wt %; Zingiber officinale of 0.04˜0.07 wt %; and Glycyrrhiza uralensis of 0.01-0.05 wt %.

### TECHNICAL FIELD

The present invention relates to a drinking water, and more particular a drinking water for a diabetic easy to manufacture and manufacturing process thereof. The drinking water include primarily Oryza sativa germinated 4 or 5 days, and secondly Zingiber officinale and Glycyrrhiza uralensis. The drinking water has a unique taste and incense for an old and feeble person having diabetes to drink effectively.

### BACKGROUND ART

Because of a pattern of dietary life in Korea being changed to meat-oriented western dietary life style, i.e. the moderns tendency toward eating fried chicken, roast pork or beef and short-order food (fastfood) made from meat to decrease a vegetable diet, lack of exercise, a greasy food contaminated with antibiotics and agrichemicals, and the modems eating habits for a roasted food and a processed food, diseases of adult people and an incurable disease, which were not present in the past times, has arisen.

Since, among the disease of adult people, diabetes which one of serious and inveterate diseases, is occurred to a young child, and the modems, concern about diabetes is increased, a quality of food is appreciated as an important one in connection with the health of body and mind.

Chemical-free foods are considered as the topics for maintaining health, since needs for environment-friendly farm produce grow with increasing speed and an importance of vegetable diet is understood.

Among the chemical-free foods, Sprouts are now recognized as new health foods, which have plenty of nutrients and are practically not contaminated with agrichemicals and fertilizers.

Sprouts mean a bud or a shoot. Growth of plant is brimming over with vitality in the bud. That is to say, it is well known that as nutrients required for sustaining life is concentrated in a germinated period, and the nutrients are appeared as a bud, the plant in the germinated period has 4 or more times effective elements such as vitamin and mineral than that of fully grown plant.

Of course, there is not any concern about agrichemicals and bacteria, because the plant in the germinated period is harvested before beginning of damages of blight and harmful insects.

Enzymes in the bud of plant are the substances differentiating between a germinated seed and a non-germinated seed. The enzymes in the bud performs functions for converting starch to starch sugar, a protein to an amino acid, and fat to fat acids, and for digesting food in a digestive organ to make nutritive elements easy to use.

### DISCLOSURE OF INVENTION

### Technical Problem

However, as the enzymes are generally not present in a cooked food or processed food, and the moderns eating habit is changed to a roasted food or instant food rather than fresh vegetables or germinated plants, lack of enzyme is aggravated.

For these reasons, the human being suffers from diabetes, cancers and degenerative diseases.

Therefore, functional foods, which can treat and prevent diabetes, are heartily required, as a bud of plant is the most ideal chemical-free food, which has all the essential nutrients for maintaining life, and lacks of stress caused by damages by blight and harmful insects.

### Technical Solution

The object of the present invention is to provide a drinking water for a diabetic easy to manufacture and manufacturing process thereof, which can treat and prevent diabetes in a very natural way through effective substances contained in a bud of Oryza sativa by providing a composition made from the bud of Oryza sativa, which has plenty of nutrients and is not contaminated, and used in a drinking water.

In order to achieve the object, a drinking water for a diabetic according to the present inventions comprises water of 93-97 wt %, budded Oryza sativa of 2.5˜5 wt %, Zingiber officinale of 0.04˜0.07 wt % and Glycyrrhiza uralensis of 0.01˜0.05 wt %.

A manufacturing process for making a drinking water according to the present invention comprises steps of: germinating step capable of producing plenty of substances effective for diabetes by germinating a bud of Oryza sativa; heating water of 93˜97 wt % with Glycyrrhiza uralensis of 0.01˜0.05 wt % at 100° C. for 60˜80 minutes; adding Zingiber officinale of 0.04˜0.07 wt % and heating the same for 20˜30 minutes; adding budded Oryza sativa of 2.5˜5 wt %, heating the same for 60˜80 minutes, and extracting the same; and filtering the heated and extracted a solid body, and containing the filtered solution in a container.

### MODE FOR THE INVENTION

A preferred embodiment of the present invention will be described in detail hereafter.

Generally, a typical food for a diabetic in market is a low calory sweetener. There are some research reports about foods effective for diabetes. For example, Nutritious drink for a light meal KIM, Hui Seon, The Korean Nutrition Society, 1994, 27(8), pp. 805-818), supplementary food for medical treatment using a dietary fiber LEE, Yeon Gyeong, The Korean Nutrition Society, 1996, 25(5), pp. 846-854), and effect of eating Polygonatum Odoratum var. Pluriflorum Ohwi to diabetes LIM, Suk Ja, The Korean Nutrition Society, 1995, 28(8), pp. 727-736) disclose the foods effective for diabetes.

Even though, research for factor of food decreasing blood-sugar, and developing of food required for a diabetic are vigorously in progress, any drinking water for a diabetic was not developed and used.

On the other hand, research using a bud of barley effective for diabetes is vigorously in progress (Japan food industrial society, 1992, 35(9), pp. P30-33), concern about regulating of vital function by using the bud is increased. However, research for relieving or curing diabetes by using the bud of barley is hardly in progress.

According to well-known data, Oryza sativa has elements such as fat, a fibroid material, lime powder, phosphorus, potassium, vitamin B1, and niacin. The content of the elements are almost twice as much as that of polished rice.

In particular, as non-polished rice has numbers of fibroid materials and a bud or oil of the rice has oryzanol, the rice can increase the function of insulin and is effective for curing diabetes.

The oryzanol is known to have outstanding effects for bettering Dyslipidemia and blood-sugar, and for decreasing serum, Trigryceride and concentration of total cholesterol. The oryzanol also can increase the amount of excretion of fat and total cholesterol, settle down an autonomic nerve, and activate a brain cell to have an effect for preventing cerebral apoplexy and dementia.

Therefore, if an adult with high probability of diabetes due to overweight or fatness eats non-polished cereal corps, sensitivity of insulin is increased to lower probability of diabetes.

Here, the term sensitivity means reactivity of the human body to insulin which is a kind of hormone storing dextrose formed in blood after a diet in a cell in the human body. The cell uses dextrose as an energy source.

On the other hand, an adult diabetic is insensible to insulin, and dextrose is not stored in a cell of human body to increase blood-sugar.

On one hand, if insulin is secreted with small quantity, sensitivity of insulin to the human body is increased, and on the other hand, if insulin is excessively secreted, sensitivity of insulin to the human body is decreased. Therefore, it is preferable for a diabetic to substitute refined cereals such as a white bread, a roll of bread, cereals, and a polished rice with a non-polished cereals.

Oryza sativa according to the present invention has an abundance of fibroid materials to slow down a rate of dextrose releasing into blood to maintain the blood-sugar level at a low level, and insulin is less needed.

Therefore, because fibroid materials and minerals helpful to health are eliminated if outer hulls of Oryza sativa is removed in the course of polishing Oryza sativa in order for making rice, the present invention uses a non-polished Oryza sativa (raw rice).

In an experiment according to the present invention, II adults, which have 27 of BMI and take hardly exercise at ordinary times, eat alternatively non-polished cereals (for example, a raw rice and a barley) and refined carbohydrates by 55% of total calorie for 6 weeks. As a result, the amount of secretion of insulin is lowered by 10% and the blood-sugar level is also decreased, when eating non-polished cereals rather than refined carbohydrates.

As will be well known from the results, the present invention provides a drinking water capable of decreasing effectively blood sugar by using non-polished cereals having an effect of lowering the amount of secretion of insulin, preferably enzymes or fibroid materials of Oryza sativa (raw rice), and other elements effective for decreasing blood sugar, and more preferably a bud of Oryza sativa (raw rice) as main elements.

In addition, a Chinese medicine material such as Zingiber officinale and Glycyrrhiza uralensis can be added to the main elements to complete the present invention.

Glycyrrhiza uralensis used in the present invention functions not only to mitigate bitterness of a Chinese medicine material but also to harmonize 72 stone medicines with 1200 herb medicines to increase the effect of medicine.

As disclosed in DONGUIBOGAM, Glycyrrhiza uralensis controls chillness and fever of the five viscera and the six entrails, normalizes physiology of an eye, a nose, the mouth, the ear, and urine and feces, smoothes the bloodstream, reinforces muscles and bone, and increases nutritive conditions and counteracts toxicity of all the medicines. Above all the things, Glycyrrhiza uralensis has been known to have a excellent effect for various kinds of toxins in combination with Zingiber officinale and a jujube.

There is not anything comparable with Glycyrrhiza uralensis in counteracting sitotoxism, medicinal poisoning, and an antitumor detoxication. Glycyrrhiza uralensis also has antagonism against various kinds of toxic medicines to cure medicinal poisoning caused by a poison, and performs counteraction function for a toxin caused by bacteria.

As described above, Glycyrrhiza uralensis is an essential sovereign remedy in the traditional oriental medicine because Glycyrrhiza uralensis counteracts a toxin from all medicines. Glycyrrhiza uralensis has a regulating function as adrenocortical hormones to relieve a pain due to muscle tension, or nervous tension, and a great effect for a gastric ulcer and a duodenal ulcer.

Further, Glycyrrhiza uralensis has a function of eliminating cholesterol to prevent arteriosclerosis, and a function of counteracting a toxin to reinforce the function of the liver. In addition, Glycyrrhiza uralensis has been known to have an obvious effect for hypopituitarism, Addison's disease, infectious hepatitis, bronchial asthma, dermatitis, malaria, chilblains and various kinds of diseases.

Zingiber officinale is used in almost half of all the traditional oriental medicine prescriptions. As disclosed in SHINONGBON CHOGYEONG, Zingiber officinale is beneficial to the human body to extract high spirits if eating Zingiber officinale continuously. The virtue of Zingiber officinale revealed by the modern medical science is to prevent an ascent of blood cholesterol strongly. Zingiber officinale has a disinfectant and antifungal function, an element for stimulating secretion of digestive fluid and quickening movement of the stomach and intestines to improve one's appetite and to help digestion. Sweet smell and hot taste elements in Zingiber officinale has been known to increase the circulation of blood and the body heat, and used as a fever remedy, and medicine for interruption in blood circulation and a cold.

Therefore, a drinking water according to the present invention uses enzymes and fibroid material of Oryza sativa (raw rice) not only to prompt the insulin function of a diabetic and to decrease the blood-sugar level but also increase energy of an old and feeble person through synergism of Zingiber officinale and Glycyrrhiza uralensis.

Hereinafter, a method for producing the drinking water according to the present invention will be described in detail.

A well-selected Oryza sativa (raw rice) is washed by clean underground water of 15˜17° C., and fully sunken in water in order to absorb enough moisture (preferably, 60˜70% of water content) required for germination.

New water is changed by 9-10 times at intervals of 4-5 hours to remove agrichemicals on a hull.

Water is remove from Oryza sativa After Oryza sativa is fully soaked. Then, Oryza sativa is placed in a Germinator (Biomax Inc. located at MUANGUN or Kwangwon farm machine Inc, located at Kimje city) and germinated at 18-20° C. for 4-5 days to germinate a sprout. At this time, a height of the sprout is about 1-5 cm.

After germinating, Oryza sativa is moved to a drying machine, which is operated by sunlight, a hot wind or electric heat, to dry.

Zingiber officinale and Glycyrrhiza uralensis, which are washed by clean underground water, and sterilized water are prepared.

When supposing that a mixture composed by Oryza sativa (germinated), Zingiber officinale and Glycyrrhiza uralensis is 100 wt %, Oryza sativa (germinated) is 98 wt %, Zingiber officinale is 1.5 wt % and Glycyrrhiza uralensis is 0.5 wt %. At this time, the mixture composed by Oryza sativa (germinated), Zingiber officinale and Glycyrrhiza uralensis is mixed with water at a ratio of 1:20.

When the ratio is converted to weight, water is 20 kg, Oryza sativa (germinated) is 0.98 kg, Zingiber officinale is 0.015 kg and Glycyrrhiza uralensis 0.005 kg.

First, Glycyrrhiza uralensis of 0.005 kg is put in water to heat at 100° C. for 60-80 minutes. Next, Zingiber officinale of 0.015 kg is added to heat for 60-80 minutes. Finally, Oryza sativa (germinated) is added to heat for 20-40 minutes, and to be extracted.

Further, the extracted resultant is filtered by a fine filter, and is contained in a container.

On the other hand, it is noted that Glycyrrhiza uralensis is put in water at the very beginning as Glycyrrhiza uralensis is very hard, and Oryza sativa (germinated) is finally put in water to prevent an element effect for diabetes from destruction after Zingiber officinale and Glycyrrhiza uralensis are mixed.

In order to check an effect of the resultant drinking water for diabetes, 10 diabetics whose blood-sugar level is over 300 mg/□ ate the drinking water of 0.5 l from day to day for 4-5 months. As a result, the blood-sugar level was approached to the normal level.

α-glucosidase in each of Oryza sativa and a sprout is depicted in the table 1. Table 1 shows that inhibitive action of the sprout is high.

TABLE 1
[Table 1] α-glucosidase in each of Oryza sativa and a sprout.

| Sample | Extracted Solution | α-glucosidase, % |
|---|---|---|
| Oryza sativa | Chloroform | 5.30 ± 0.09 |
| | Hexane | 7.02 ± 1.16 |
| | Ethyl acetate | 12.24 ± 0.08 |
| | Water | 20.31 ± 2.16 |
| Sprout | Chloroform | 12.70 ± 2.08 |
| | Hexane | 62.93 ± 1.09 |
| | Ethyl acetate | 48.26 ± 2.09 |
| | Water | 40.76 ± 0.07 |

According to analysis result by HPLC for Hexane in each of Oryza sativa and sprout, more materials were formed in case of sprout as shown in tables 2 and 3.

TABLE 2
HPLC for Oryza sativa

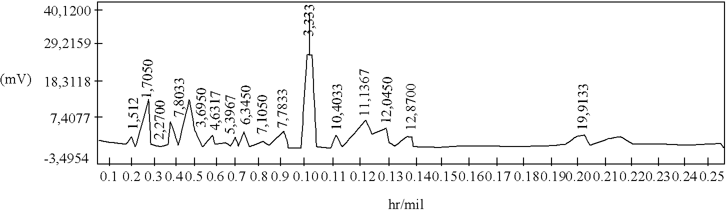

TABLE 3
HPLC for sprout

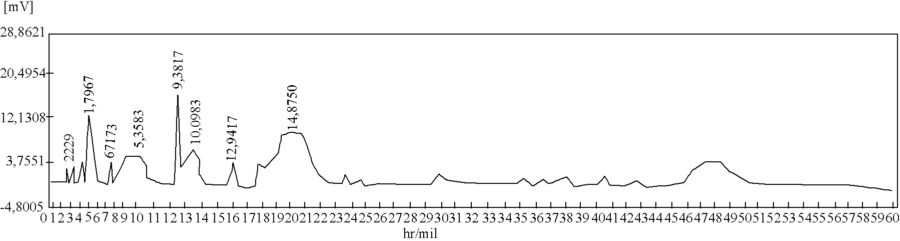

By analyzing a delaying effect for sugar-dialysis, the delaying effect is high for sprout as shown in table 4.

TABLE 4
Delaying effect for sugar-dialysis in each of Oryza sativa and sporut.

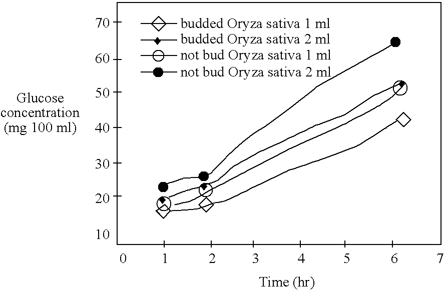

### INDUSTRIAL APPLICABILITY

The drinking water according to the present invention uses enzymes and fibroid material of Oryza sativa (raw rice) not only to prompt the insulin function of a diabetic and to decrease the blood-sugar level but also increase energy of an old and feeble person through synergism of Zingiber officinale and Glycyrrhiza uralensis.

1. A drinking water for a diabetic comprising: water of 93˜97 wt %; budded Oryza sativa of 2.5˜5 wt %; Zingiber officinale of 0.04˜0.07 wt %; and Glycyrrhiza uralensis of 0.01-0.05 wt %.

2. A manufacturing process for making a drinking water comprising the steps of: germinating Oryza sativa; heating water of 93˜97 wt % with Glycyrrhiza uralensis of 0.01-0.05 wt % at 100° C. for 60-80 minutes; adding Zingiber officinale of 0.04-0.07 wt % and heating for 20-30 minutes; adding budded Oryza sativa of 2.5-5 wt %, heating for 60-80 minutes and extracting; and filtering the heated and extracted solid body, and containing the filtered solution in a container.

3. A manufacturing process according to claim 2, wherein in the germinating step, Oryza sativa is washed by clean water of 15-17° C. and fully sunken in water to absorb enough moisture, and the Oryza sativa is germinated in a Germinator at 18-20° C. for 4-5 days to germinate a sprout, a height of the sprout being about 1-5 cm.

4. A manufacturing process according to claim 2, wherein the water is changed by 9-10 times at intervals of 4-5 hours.

5. A manufacturing process employing the water according to claim 1, and comprising mixing a mixture composed by Oryza sativa (germinated), Zingiber officinale and Glycyrrhiza uralensis with the water at a ratio of 1:20.
